(19) United States
(12) Patent Application Publication
(10) Pub. No.: US 2021/0154341 A1
(43) Pub. Date: May 27, 2021

(54) METHOD AND APPARATUS FOR STERILIZING AN ARTICLE BY MEANS OF A PULSED LIGHT SOURCE

(71) Applicant: EbeTech GmbH, Scheessel (DE)

(72) Inventor: Mirko Ebeling, Scheessel (DE)

(73) Assignee: EbeTech GmbH, Scheessel (DE)

(21) Appl. No.: 16/613,505

(22) PCT Filed: May 7, 2018
(86) PCT No.: PCT/EP2018/000220

§ 371 Date: Nov. 14, 2019

(30) Foreign Application Priority Data
May 18, 2017 (DE) 10 2017 004 752.9

(51) Int. Cl. A61L 2/08

(52) U.S. Cl. CPC A61L 2/084; A61L 2202/11; A61L 2202/21; A61L 2202/14; A61L 2202/122

(57) ABSTRACT

A method and a device for sterilizing an object for a pharmaceutical or cosmetic application in a decontaminated environment, wherein the object is transported from a first region to a second region through a lock and after the sterilization has been carried out it is further transported for further handling into a third region through a further lock. The object is completely irradiated from all sides by pulsed light in the second region for the sterilization.

### CROSS REFERENCE TO RELATED APPLICATIONS

This application is the US National Phase of and claims the benefit of and priority on International Application No. PCT/EP2018/000220 having an international filing date of 7 May 2018, which claims priority on German Patent Application No. 10 2017 004 752.9 having a filing date of 18 May 2017.

### BACKGROUND OF THE INVENTION

### Technical Field

The invention relates to a method for sterilizing an object for a pharmaceutical or cosmetic application in a decontaminated environment, wherein the object is transported from a first region to a second region through a lock and after the sterilization has been carried out it is further transported for further handling into a third region through a further lock. Furthermore, the invention relates to a device for sterilizing an object for a pharmaceutical or cosmetic application having a decontaminated chamber, with locks for bringing the object into or out from the chamber and with a means of transport for transporting the object into and out from the chamber.

### Prior Art

In the production of pharmaceutical as well as cosmetic products, the highest demands are placed on the cleanliness and sterility not only of the objects used, such as packages, containers, syringes, vials, and the like, but also on the means of handling the objects. Even the slightest amount of microorganisms and/or germs which has become deposited for example in a syringe for pharmaceutical application may result in severe health injuries. The same holds for germs getting onto a package for such objects. Thus, for example, by cross contamination, a contamination of a package or the like may result in a contamination of the packaged object.

Methods or techniques and devices are known for the sterilization of pharmaceutical and cosmetic objects, during which the objects for example are exposed to a gas such as hydrogen peroxide. By the “washing” of the objects with hydrogen peroxide, at least the greater portion of all germs can be killed. However, the expense for building up a hydrogen peroxide atmosphere in a treatment chamber as well as the venting of the chamber in which the sterilization occurs is very time and cost intensive. Furthermore, areas at which the objects are held during the sterilization by holding means or standing surfaces are not washed by the gas.

Furthermore, it is known how to irradiate objects being sterilized with an e-beam. In this case, the object is specifically exposed to electrons, which kill the germs by their ionizing action. However, since the safety expense in this method for the operation of such a device is very high, this method of sterilization also turns out to be especially costly and expensive.

### BRIEF SUMMARY OF THE INVENTION

Starting from this, the problem which the invention proposes to solve is to create a method as well as a device for the sterilizing of an object by which an object can be sterilized in an especially simple and favorable manner.

A method for solving this problem is a method for sterilizing an object, especially a tub, for a pharmaceutical or cosmetic application in a decontaminated environment, wherein the object is transported from a first region to a second region through a lock and after the sterilization has been carried out it is further transported for further handling into a third region through a further lock, characterized in that the object is completely irradiated from all sides by pulsed light or electromagnetic waves of a light source or radiation source in the second region for the sterilization, wherein the object in the second region is automatically grabbed at least once by a robot arm, oriented by the robot arm at least once relative to the light source for the irradiation, and again set down, and the object is grabbed several times by the robot arm at various positions, oriented relative to the light source and set down again until the object has been completely exposed to the light or electromagnetic waves of the light source and wherein after an irradiation and a setting down of the object by the robot arm and before a further grabbing of the object or of a further object the robot arm is likewise sterilized by the light source.

Accordingly, it is provided, for sterilizing an object, especially a tub, for a pharmaceutical or cosmetic application in a decontaminated environment, to transport the object from a first region to a second region in which the sterilization occurs. In a third region a further production process may take place, such as the loading of the object with further means. The different regions are separated from each other by locks. In the second region, the object is completely irradiated from all sides by light or pulsed electromagnetic waves of a light source or radiation source for the sterilization, wherein the object in the second region is automatically grabbed at least once by a robot arm, oriented by the robot arm at least once relative to the light source for the irradiation, and again set down, and the object is grabbed several times by the robot arm at various positions, oriented relative to the light source and set down again until the object has been completely exposed to the light of the light source and wherein after an irradiation and a setting down of the object by a robot arm and before a further grabbing of the object or of a further object the robot arm is likewise sterilized by the light source. Because of the fact that the object is irradiated on all sides, no shadow is formed in which germs might survive. Thanks to the use of a light source which preferably emits light of the visible optical spectrum, no elevated safety measures are necessary for the use of this light source. Also the exposing of the object to light, especially light of the visible optical spectrum, proves to be especially fast and easy, since it is not necessary to first produce a given concentration of a gas in the second region. Thus, an especially simple and favorable way of sterilization of an object can be realized by the use of the pulsed light or the pulsed electromagnetic radiation for the sterilization. After the object has at first been grabbed by the robot arm and oriented variously relative to the light source for the irradiation, the object is set down again, in order to be grabbed in a different position, so that the regions where the object was previously grabbed by the robot arm can also be exposed to light. In this way, the object can be completely exposed to light for the sterilization. Therefore, no areas remain where germs can survive.

Preferably, it is further provided that the object is automatically grabbed at least once by a robot arm in the second region, oriented by the robot arm several times relative to the light source for the irradiation depending on the shape of the object, and again set down. After the object has been transported through the lock from the first region to the second region, the object is grabbed in fully automated manner by a robot arm and oriented relative to the light source, which is situated inside or outside the second region, so that the object is exposed to light as completely as possible. After the first irradiation of the object by the light source or the radiation, the object is reoriented by a robot arm so that areas of the object formerly lying in the shadow can likewise be reached by further irradiation. After the irradiation is completed, the object is set down by the robot once more. Since generally the same objects are always irradiated, the robot arm can perform the sterilization of the object fully automated in concert with the light source. In the event that another object needs to be sterilized, the robot arm can be reprogrammed in known manner in order to also expose this object to light in an adequate manner.

A further advantageous embodiment of the present invention may provide that the object is irradiated by the light source with light pulses of different rate, pulse duration, intensity, energy and/or repetition rate in dependence on the application and contamination of the object. Thus, for example, it may be provided that pharmaceutical products are exposed with a higher intensity, pulse duration, energy or repetition rate of the light pulse than objects for a cosmetic application. Likewise, it is conceivable that products which themselves may react sensitively to light or electromagnetic radiation are exposed to the light with a lower intensity. In these cases, it is conceivable to increase the pulse duration or the rate, so as to assure that at least the greater portion of all germs have been hit. For especially heavily contaminated or contamination-prone objects, a special light treatment may also be designed. The values for these properties of the light pulse can be set manually or be chosen automatically by a control unit upon detecting the object. Thanks to this automated setting of the quality or quantity of the irradiation, an adequate irradiation of the objects can be ensured.

Furthermore, it may be provided according to the invention that after an irradiation and a setting down of the object by a robot arm and before a further grabbing of the object or of a further object the gripping means of the robot arm are likewise sterilized by the light source. For the grabbing of the object, the robot arm comprises grasping means, which may be configured for example as a grip, suction cups, or also electromagnets. Before the grabbing of an object, the grasping surfaces of these grasping means are also exposed to the sterilizing light by the light source, so that here as well no germs can take hold. Thus, thanks to the sterilization of the grasping means, a cross contamination between the robot arm and the objects is avoided. The exposure of the grasping means to the light may depend on the type of grasping means. Thus, for example, the grasping means can also be exposed to the light from various directions by corresponding orienting of the robot arm.

Furthermore, it may be provided that a pressure gradient, especially a pressure cascade, is organized between the different regions by the locks. Thanks to this pressure cascade, it can be guaranteed that no contamination is sucked into the region in which the sterilization occurs. By maintaining an excess pressure in the second region with respect to the other regions, all foreign substances can be forced out from the second region. The second region may be, for example, an isolator, which is decontaminated for example with hydrogen peroxide after completion of the sterilization of a batch of objects. In order to verify that an adequate decontamination has taken place, bioindicators for example can be supplied to the second region, which will detect any residual contamination. Furthermore, for a checking of the quality of the electromagnetic radiation or the light, a bioindicator carrying germs can be exposed to the light at regular intervals. By observing the growth of the irradiated germs it can be ascertained whether the light source still provides a sufficiently high power to kill the germs.

In particular, the present invention may provide that the objects are tubs for the holding of glass containers. The tubs are unpacked in the first region, sterilized in the second region, preferably an isolator, and the glass containers after the unpacking are provided with a means for the pharmaceutical or cosmetic application in a third region. The tubs may be tray or box like objects, whose opening is closed for example by a film. However, it is also conceivable to sterilize any other objects for the pharmaceutical or cosmetic application by the described method.

A device for the solving of the aforementioned problem is a device for sterilizing an object fora pharmaceutical or cosmetic application having a decontaminated chamber, with locks for bringing the object into or out from the chamber and with a means of transport for transporting the object into and out from the chamber, characterized in that the chamber is associated with a light source for pulsed light or for pulsed electromagnetic waves for the sterilizing and complete irradiation of the object from all sides.

Accordingly, a device for sterilizing an object for a pharmaceutical or cosmetic application is provided having a decontaminated chamber, with locks for bringing the object into or out from the chamber and with a means of transport for transporting the object into and out from the chamber. The chamber is associated with a light or radiation source for pulsed light or for pulsed electromagnetic waves, which is designed for the sterilizing and complete irradiation of the object. Thanks to the pulsed light, all germs on the object can be killed. Thanks to the irradiation on all sides, no shadow sides occur during the irradiation where the germs might survive. Thus, thanks to this irradiation of the objects with light, a complete sterilization of the objects can be realized in a simple and favorable manner.

Preferably, it may be provided by the present invention that a robot arm is positioned in the chamber for the grabbing of the object, for the relative orienting of the object to the light source, and for setting down the object, wherein the specimen can be repeatedly grabbed, oriented and set down in the chamber for a complete irradiation. The robot arm is controlled in this process by a control device. Depending on the type and form of the object, as well as the position of the light source and type of light source, the robot arm can be programmed so that the entire object can be sterilized by the light. The robot arm may have a plurality of axes in order to perform any movement needed in order to orient the object relative to the light source. The robot arm also fulfills all the requirements for working in a sterile environment. The light source may be arranged either in the chamber for the sterilization or outside the chamber. In the event that the light source is arranged outside the chamber, a transparent window pane should be used, which absorbs the light for the sterilizing of the object only to a slight extent.

For the grabbing of the object, the robot arm is coordinated with an effector or a grasping means, which may be configured as a grip, a suction cup, or also a magnet.

After the object is set down by the robot arm, possibly after several irradiation cycles, the object can be taken by a transport means, preferably a conveyor belt, out through the lock and taken to a further chamber. It is also conceivable that the lock is opened after the end of the irradiation and the object is placed by the robot arm directly in the following chamber.

Preferably, it may furthermore be provided by the present invention that the object can be irradiated by the light source with light pulses of different rate, pulse duration, intensity, energy and/or repetition rate in dependence on the application and contamination of the object. The quality and the quantity of the light or the radiation can be set by the control device. The choice of the different parameters for the light can be set manually on the control device or automatically by the control device.

### BRIEF DESCRIPTION OF THE DRAWINGS

A preferred exemplary embodiment of the invention shall now be explained more closely with the aid of the drawing. This shows:

FIG. 1 is a perspective view of a device for the sterilizing of an object;

FIG. 2 is another perspective view of the device of FIG. 1;

FIG. 3 is a view of the device of FIG. 1;

FIG. 4 is a cross sectional representation through the device of FIG. 1; and

FIG. 5 a view of the device of FIG. 1 without housing.

### DETAILED DESCRIPTION OF PREFERRED EMBODIMENTS

For the sterilizing of objects for a pharmaceutical or cosmetic application, the objects are taken through a device 10 and exposed to pulsed light according to the invention.

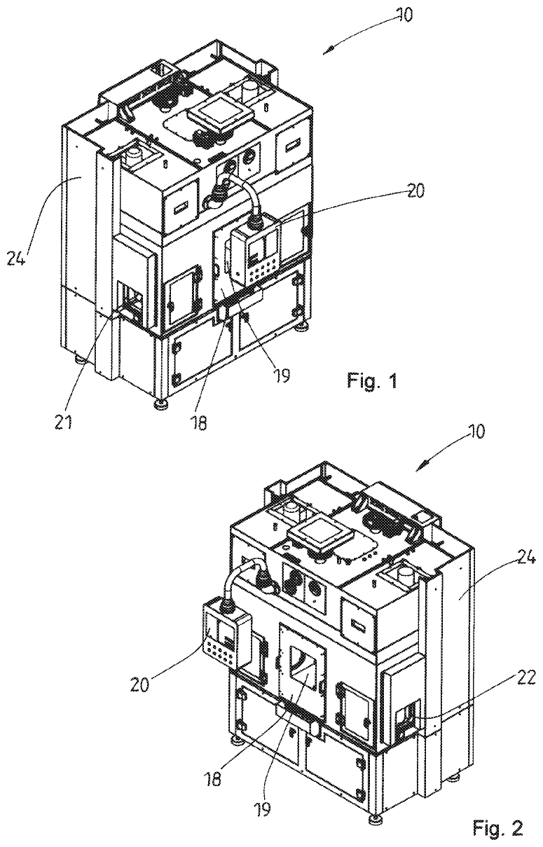

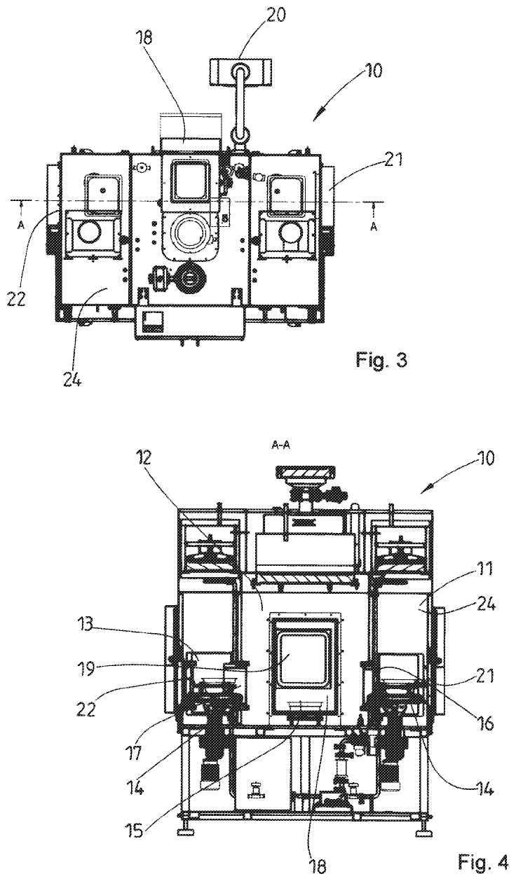

The device 10 for the sterilizing of objects consists, in the exemplary embodiments represented in FIGS. 1 to 4, of a first region 11, a second region 12 and a third region 13 (FIG. 4). These three regions 11, 12, 13 may each be designed as closed chambers. Thus, for example, it is provided that the second chamber is formed as an isolator, which can be decontaminated by corresponding cycles.

While the first region 11 and the second region 12 each have a conveyor belt 14, a stand 15 is provided at the center of the second region 12, for the at least temporary standing of the object. Between the first region 11 and the second region 12 and between the second region 12 and the third region 13 there is provided a respective lock 16, 17, which hermetically separates the second region 12 from the first region 11 and the third region 13.

According to the invention, the middle region 12 is associated with a robot arm 23. This robot arm 23 can manipulate the object being sterilized. Furthermore, a light or radiation source (not shown) is associated with the second region 12. This light source may either be arranged in the region 12 or outside the region 12. In the event that the light source is arranged outside the second region 12, the source is arranged on a door 18 to the second region 12 in such a way that the light or the electromagnetic waves of the light source can shine through a window 19 into the interior of the second region 12. The window 19 has a high transmission rate for the light or the corresponding electromagnetic radiation of the light source.

Both the robot arm 23 and the light source can be actuated by a control device. The control can occur manually through an input device 20 or automated by the control device.

Furthermore, the device 10 comprises additional devices for ventilation of the different regions 11, 12, 13, as well as two further locks 21, 22 for loading the first region 11 and for releasing the object from the third region 13.

The device represented in the figures is accommodated in a compact, box-like frame with a housing 24. However, it is also conceivable for the device 10 to be integrated in another installation or to consist of individual parts flanged together.

For the sterilizing of the object (not shown), the object is supplied through a lock 21 to the first region 11. Here, the still packaged object can be unpacked, for example. After this, the lock 16 opens and the robot arm 23 removes the object from the first region 11, so as to then orient it relative to the light source for the irradiation. After the pulsed irradiation has occurred, the orientation of the object relative to the light source can be changed again by the robot arm 23, so that formerly shadowed areas of the object can also be exposed to the light. Once the entire surface of the object has been irradiated in this way, the object can be set down on the stand 15 and the grasping means of the robot arm 23 are likewise sterilized by the light source. The now sterilized grasping means of the robot arm 23 then grabs the object once more, at positions already sterilized by exposure to the light. In the following, the regions where the grasping means of the robot arm 23 were applied are then also sterilized by the light source. In the next step, the lock 17 to the third region 13 is opened, whereupon the robot arm 23 sets the object down on the conveyor belt 14 in the third region 13. So that a contamination of the second region 12 does not occur during the entering and leaving through the locks, an excess pressure prevails in the second region 12 relative to the regions 11 and 13, so that air is forced out of the second region 12 into the regions 11 and 13 and a contamination is prevented. After the sterilized object has been set down in the third region 13, the lock 17 closes and the object is ready for a further processing step.

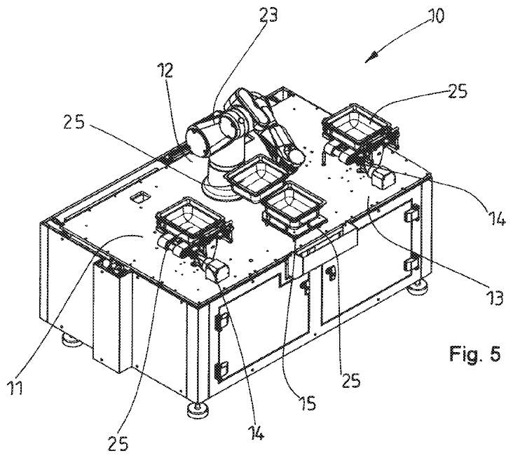

FIG. 5 shows the device without the housing 24 for purposes of illustration. The robot arm 23, which is arranged in the second region 12, or in the middle chamber, has grabbed a tub 25 by means of its grasping means for the holding of further objects and orients this relative to the light source, not shown. Thanks to the plurality of axes of the robot arm 23, it can move into all necessary positions relative to the light source. Further tubs 25 are situated on the conveyor belts 14 in the regions 11 and 13 and also on the stand 15.

At regular intervals, the second region 12, or the isolator, can be decontaminated by rinsing with hydrogen peroxide, for example. For checking the degree of contamination or decontamination, a bioindicator can be supplied to the second region 12. Likewise, the radiation power of the light source should be checked at regular intervals by means of a bioindicator.

It is expressly pointed out that the present invention is not limited to the exemplary embodiment represented by the drawing. Instead, it is provided that the invention may also be realized by other exemplary embodiments.

### LIST OF REFERENCE NUMBERS

10 Device

11 First region

12 Second region

13 Third region

14 Conveyor belt

15 Stand

16 Lock

17 Lock

18 Door

19 Window

20 Input device

21 Lock

22 Lock

23 Robot arm

24 Housing

25 Tub

1. A method for sterilizing an object, especially a tub (25), for a pharmaceutical or cosmetic application in a decontaminated environment, wherein the object is transported from a first region (11) to a second region (12) through a lock (16) and after the sterilization has been carried out it is further transported for further handling into a third region (13) through a further lock (17), comprising completely irradiating the object from all sides by pulsed light or electromagnetic waves of a light source or radiation source in the second region (12) for the sterilization, wherein the object in the second region (12) is automatically grabbed at least once by a robot arm (23), oriented by the robot arm (23) at least once relative to the light source for the irradiation, and again set down, and the object is grabbed several times by the robot arm (23) at various positions, oriented relative to the light source and set down again until the object has been completely exposed to the light or electromagnetic waves of the light source and wherein after an irradiation and a setting down of the object by the robot arm (23) and before a further grabbing of the object or of a further object the robot arm (23) is likewise sterilized by the light source.

2. The method for sterilizing an object as claimed in claim 1, wherein the object is automatically grabbed at least once by the robot arm (23) in the second region (12), oriented by the robot arm (23) several times relative to the light source for the irradiation depending on the shape of the object, and again set down.

3. The method for sterilizing an object as claimed in claim 2, wherein the object is grabbed several times by the robot arm (23) at various positions, oriented relative to the light source and set down again until the object has been completely exposed to the light of the visible optical spectrum of the light source.

4. The method for sterilizing an object as claimed in claim 1, wherein the object is irradiated by the light source with light pulses of different rate, pulse duration, intensity, energy and/or repetition rate in dependence on the application and contamination of the object.

5. The method for sterilizing an object as claimed in claim 1, wherein after an irradiation and a setting down of the object by the robot arm (23) and before a further grabbing of the object or of a further object the gripping means of the robot arm (23) are likewise sterilized by the light source.

6. The method for sterilizing an object as claimed in claim 1, wherein a pressure gradient, especially a pressure cascade, is organized between the different regions by the locks (16, 17).

7. The method for sterilizing an object as claimed in claim 1, wherein the objects are tubs (25) for the holding of glass containers, the tubs (25) are unpacked in the first region (11), sterilized in the second region (12), preferably an isolator, and the automatically unpacked glass containers are provided with a means for the pharmaceutical or cosmetic application in a third region (13).

8. A device (10) for sterilizing an object for a pharmaceutical or cosmetic application having a decontaminated chamber, with locks (16, 17) for bringing the object into or out from the chamber and with a means of transport for transporting the object into and out from the chamber, comprising a light source for pulsed light or for pulsed electromagnetic waves for the sterilizing and complete irradiation of the object from all sides, wherein the chamber is associated with the light source.

9. The device (10) for sterilizing an object as claimed in claim 8, further comprising a robot arm (23) is positioned in the chamber for the grabbing of the object, for the relative orienting of the object to the light source, and for setting down the object, wherein the object can be repeatedly grabbed, oriented and set down in the chamber for a complete irradiation.

10. The device (10) for sterilizing an object as claimed in claim 8, wherein the object can be irradiated by the light source with light pulses of different rate, pulse duration, intensity, energy and/or repetition rate in dependence on the application and contamination of the object.

11. The device (10) for sterilizing an object as claimed in claim 9, wherein the object can be irradiated by the light source with light pulses of different rate, pulse duration, intensity, energy and/or repetition rate in dependence on the application and contamination of the object.
